(19) United States
(12) Patent Application Publication
(10) Pub. No.: US 2024/0254072 A1
(43) Pub. Date: Aug. 1, 2024

(54) DERIVATIVES OF CANNABIDIOL-C4 FOR THE TREATMENT OF EPILEPSY

(71) Applicant: GW Pharma Limited, Cambridgeshire (GB)

(72) Inventors: Alan James SILCOCK, Cambridge (GB); Karen Ka-Yen TSE, Cambridge (GB)

(21) Appl. No.: 18/560,337

(22) PCT Filed: May 11, 2022
(86) PCT No.: PCT/GB2022/051199

§ 371 Date: Nov. 10, 2023

(30) Foreign Application Priority Data
May 12, 2021 (GB) 2106788.9

(51) Int. Cl. C07C 62/32; A61K 31/00; A61K 45/06; A61P 25/08; C07C 39/23

(52) U.S. Cl. CPC C07C 62/32; A61K 31/658; A61K 45/06; A61P 25/08; C07C 39/23

(57) ABSTRACT

The present invention relates to compounds that are pharmaceutically active and methods of preparation thereof. The compounds of the invention are 7-hydroxy-cannabidol-C4 (7-OH-CBD-C4) and 7-carboxy-cannabidiol-C4 (7-COOH-CBD-C4). The compounds of the invention are related to cannabidiol (CBD). CBD is a non-psychoactive cannabinoid which has been used to treat various diseases and disorders. While such treatments hold promise, there remains a need in the art for more effective treatments and this has been brought about by way of the compounds of the invention.

### RELATED APPLICATIONS

The present application is related to, and claims the benefit of, GB 2106788.9 filed on 12 May 2021 (12.05.2021), the contents of which are hereby incorporated by reference in their entirety.

### FIELD OF THE INVENTION

The present invention relates to novel compounds that are pharmaceutically active and methods of preparation thereof. In particular, the present invention relates to 7-hydroxy-cannabidol-C4 (7-OH-CBD-C4) and 7-carboxy-cannabidiol-C4 (7-COOH-CBD-C4) and their use in the treatment of epilepsy.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE).

Intractable or treatment-resistant epilepsy was defined in 2009 by the International League Against Epilepsy (ILAE) as “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment resistant. Children who undergo frequent seizures in childhood are often left with neurological damage which can cause cognitive, behavioral and motor delays.

Childhood epilepsy is a relatively common neurological disorder in children and young adults with a prevalence of approximately 700 per 100,000. This is twice the number of epileptic adults per population.

When a child or young adult presents with a seizure, investigations are normally undertaken in order to investigate the cause. Childhood epilepsy can be caused by many different syndromes and genetic mutations and as such diagnosis for these children may take some time.

The main symptom of epilepsy is repeated seizures. In order to determine the type of epilepsy or the epileptic syndrome that a patient is suffering from an investigation into the type of seizures that the patient is experiencing is undertaken. Clinical observations and electroencephalography (EEG) tests are conducted and the type(s) of seizures are classified according to the ILEA classification.

Generalized seizures, where the seizure arises within and rapidly engages bilaterally distributed networks, can be split into six subtypes: tonic-clonic (grand mal) seizures; absence (petit mal) seizures; clonic seizures; tonic seizures; atonic seizures and myoclonic seizures.

Focal (partial) seizures where the seizure originates within networks limited to only one hemisphere, are also split into sub-categories. Here the seizure is characterized according to one or more features of the seizure, including aura, motor, autonomic and awareness/responsiveness. Where a seizure begins as a localized seizure and rapidly evolves to be distributed within bilateral networks this seizure is known as a bilateral convulsive seizure, which is the proposed terminology to replace secondary generalized seizures (generalized seizures that have evolved from focal seizures and are no longer remain localized).

Focal seizures where the subject's awareness/responsiveness is altered are referred to as focal seizures with impairment and focal seizures where the awareness or responsiveness of the subject is not impaired are referred to as focal seizures without impairment.

Cannabidiol (CBD), a non-psychoactive derivative from the cannabis plant, has demonstrated anti-convulsant properties in several anecdotal reports, pre-clinical and clinical studies both in animal models and humans. Three randomized control trials showed efficacy of the purified pharmaceutical formulation of CBD in patients with Dravet and Lennox-Gastaut syndrome.

Based on these three trials, a botanically derived purified CBD preparation (Epidiolex®) was approved by the FDA in June 2018 and the EMA in September 2019 for the treatment of seizures associated with Dravet and Lennox-Gastaut syndromes. In July 2020, the FDA approved the drug for the treatment of seizures associated with Tuberous Sclerosis Complex.

Previously, the applicant has shown that the compounds 7-hydroxy-cannabidiol (7-OH-CBD) and 7-hydroxy-cannabidivarin (7-OH-CBDV) display anticonvulsant activity in EP3160457. The data provided therein demonstrates the anticonvulsant effects of such compounds in the pentylenetetrazole-induced (PTZ) model of generalised seizures and the Maximal Electroshock (MES) model of generalised seizures.

The synthetic production of 7-hydroxy-cannabidiol (7-OH CBD) is disclosed in WO 01/95899 in addition to many other CBD derivatives. The compound was tested in a model of inflammation and found to be effective. The application goes on to suggest that the compound may be of use as an analgesic, anti-anxiety, anti-convulsant, neuroprotective, anti-psychotic and anti-inflammatory based on the mechanisms the compound displays in the model of inflammation.

Cannabidiol-C4 (CBD-C4), also known as nor-cannabidiol is a homolog of CBD, with the side-chain shortened by one methylene bridge. It is a naturally occurring cannabinoid that can be found in minor quantities in the cannabis plant. The cannabinoid can alternatively be produced by synthetic means.

In WO 2020/104796 the applicant disclosed that the compound CBD-C4 exhibits anti-convulsant activity. Therapeutic efficacy was shown in a MES model of generalised seizure, whilst a range of toxicology screens proved acceptable toxicology of the compound.

The present invention has been devised in light of these considerations.

### BRIEF SUMMARY OF THE INVENTION

At its most general, the present invention relates to the surprising discovery that the novel compounds 7-OH-CBD-C4 and 7-COOH-CBD-C4 are biologically active and hence useful in the treatment of diseases. Such novel compounds may be administered by a wide variety of routes including but not limited to oral, transdermal, buccal, nasal, pulmonary, rectal or ocular. Such compounds may be used for the treatment or prevention of a medical conditions such as epilepsy.

In a first aspect of the invention there is provided a compound of formula (I), or a salt thereof:

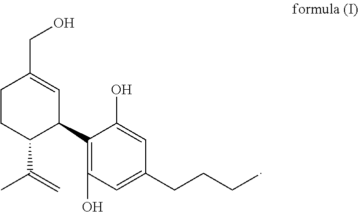

In one embodiment, the compound of the first aspect is a pure, isolated or synthetic compound.

In a second aspect of the invention, there is provided a pharmaceutical composition comprising a compound of formula (I) or a salt thereof.

In one embodiment, the pharmaceutical composition of the second aspect comprises one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

In one embodiment, the pharmaceutical composition of the second aspect is in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.

In a third aspect of the invention there is provided a compound of formula (I) or a salt thereof, for use a medicament, such as a medicament for the treatment of epilepsy.

In a fourth aspect of the invention there is provided a compound of formula (I) or a salt thereof, for use in a method of treatment, such as a method of treating epilepsy.

In a fifth aspect of the invention there is provide a method of treatment comprising administering to a subject in need of treatment a therapeutically effective amount of a compound of formula (I) or a salt thereof.

In one embodiment, the compound of formula (I) or salt thereof is used in combination with one or more concomitant anti-epileptic drugs (AEDs).

In one embodiment, the dose of the compound of formula (I) is between 1 and 2,000 mg/kg.

In accordance with a sixth aspect of the present invention there is provided a process for the production of a compound of formula (I) comprising the following steps:
i) treating 3,5-dimethoxybenzyl bromide with n-propylmagnesium chloride to produce 1 butyl-3,5-dimethoxybenzene;
ii) treating 1-butyl-3,5-dimethoxybenzene with boron tribromide to produce 5 butylbenzene-1,3-diol;
iii) coupling 5-butylbenzene-1,3-diol with (R)-isolimonenediol 7-O-acetate in the presence of catalytic p-toluenesulfonic acid to produce 7-acetoxy-CBD-C4; and
iv) treating 7-acetoxy-CBD-C4 sodium borohydride to produce the compound of formula (I).

In a seventh aspect of the invention there is provided an intermediate formed in the process of the production of a compound of formula (I), wherein the intermediate is selected from:

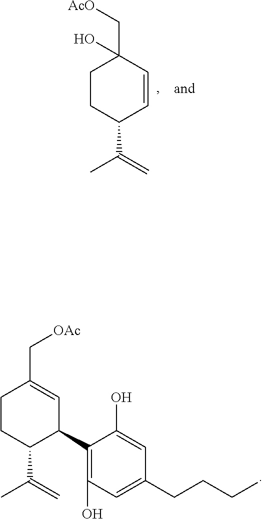

In an eighth aspect of the invention there is provided a compound of formula II or a salt thereof:

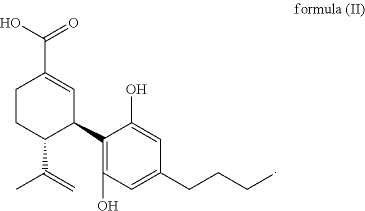

In one embodiment the compound of formula (II) is a pure, isolated or synthetic compound.

In a ninth aspect of the present invention there is provided a pharmaceutical composition comprising a compound of formula (II) or a salt thereof.

In one embodiment, the pharmaceutical composition of the ninth aspect comprises one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

In one embodiment, the pharmaceutical composition of the ninth aspect is in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.

In a tenth aspect of the invention there is provided a compound of formula (II) or a salt thereof, for use a medicament, such as a medicament for the treatment of epilepsy.

In a fourth aspect of the invention there is provided a compound of formula (II) or a salt thereof, for use in a method of treatment, such as a method of treating epilepsy.

In a fifth aspect of the invention there is provide a method of treatment comprising administering to a subject in need of treatment a therapeutically effective amount of a compound of formula (II) or a salt thereof.

In one embodiment, the compound of formula (II) or salt thereof is used in combination with one or more concomitant anti-epileptic drugs (AEDs).

In one embodiment, the dose of the compound of formula (II) is between 1 and 2,000 mg/kg.

In a thirteenth aspect of the invention there is provided a process for the production of a compound of formula (II) comprising the following steps:
i) treating 3,5-dimethoxybenzyl bromide with n-propylmagnesium chloride to produce 1-butyl-3,5-dimethoxybenzene;
ii) treating 1-butyl-3,5-dimethoxybenzene with boron tribromide to produce 5-butylbenzene-1,3-diol;
iii) coupling 5-butylbenzene-1,3-diol with (R)-isolimonenediol 7-O-acetate in the presence of catalytic p-toluenesulfonic acid to produce 7-acetoxy-CBD-C4;
iv) treating 7-acetoxy-CBD-C4 sodium borohydride to produce 7-hydroxy-CBD-C4;
v) treating 7-hydroxy-CBD-C4 with acetic anhydride and caesium carbonate to give 7-hydroxy-CBD-C4 di-O-acetate;
vi) reacting 7-hydroxy-CBD-C4 di-O-acetate with manganese dioxide to give 7-formyl-CBD-C4 di-O-acetate;
vii) oxidising 7-formyl-CBD-C4 di-O-acetate to afford 7-carboxy-CBD-C4 di-O-acetate; and
viii) deprotection of carboxy-CBD-C4 di-O-acetate using sodium borohydride to produce the compound of formula (II).

In a fourteenth aspect of the invention there is provided an intermediate formed in the process of the production of a compound of formula (II), wherein the intermediate is selected from:

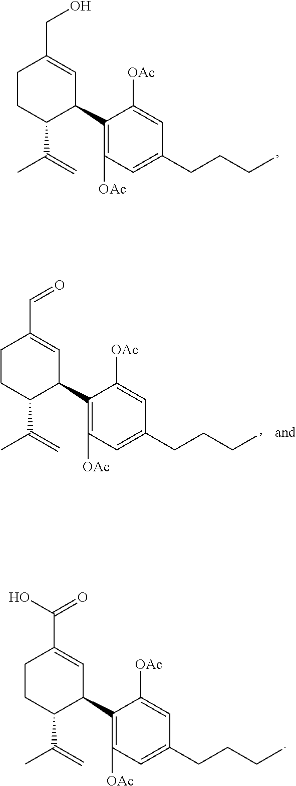

These and other aspects and embodiments of the invention are described in further detail below.

### BRIEF SUMMARY OF THE DRAWINGS

There present invention is described with reference to the figures listed below:

FIG. 1 shows the evaluation of the test compounds, shown as Compound I and Compound II, in the mini-MEST test in the mouse as described in Example 2.

### DETAILED DESCRIPTION OF THE INVENTION

The present invention relates to relates to the compounds 7-OH-CBD-C4 and 7-COOH-CBD-C4, which are biologically active and hence useful in the treatment of diseases.

### Compound I

The invention provides a compound of formula (I):

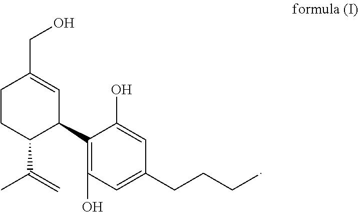

The compound of formula (I) is (1′R,2′R)-4-butyl-5′-(hydroxymethyl)-2′-(prop-1-en-2-yl)-1′,2′,3′,4′-tetrahydro-[1,1′-biphenyl]-2,6-diol. It will be referred as the compound of formula (I) or compound I herein.

### Methods of Synthesis

The invention provides a process for the production of a compound of formula (I) comprising the following steps:
i) treating 3,5-dimethoxybenzyl bromide with a propyl Grignard reagent to produce 1-butyl-3,5-dimethoxybenzene;
ii) treating 1-butyl-3,5-dimethoxybenzene with a demethylation reagent to produce 5-butylbenzene-1,3-diol;
iii) coupling 5-butylbenzene-1,3-diol with (R)-isolimonenediol 7-O-acetate in the presence of acid to produce 7-acetoxy-CBD-C4; and
iv) treating 7-acetoxy-CBD-C4 with a reducing agent to produce the compound of formula (I).

Suitable Grignard reagents include n-propylmagnesium chloride and n-propylmagnesium bromide. In preferred embodiments, n-propylmagnesium chloride is used.

Suitable demethylation reagents include boron tribromide, beryllium dichloride, aluminium trichloride, trimethylsilyliodide and pyridine hydrochloride. In preferred embodiments, include boron tribromide is used.

Suitable acids include Brønsted acids and Lewis acids. Examples of suitable Brønsted acids include p-toluenesulfonic acid. Examples of suitable Lewis acids include aluminium trichloride. In preferred embodiments, p-toluenesulfonic acid is used, such as catalytic p-toluenesulfonic acid.

Suitable reducing agents include lithium aluminium hydride, sodium bis(2-methoxyethoxy)aluminium hydride (red-AI), diborane and sodium borohydride. In preferred embodiments, sodium borohydride is used.

### Intermediates

The invention provides an intermediate formed in the process of the production of a compound of formula (I), wherein the intermediate is selected from:

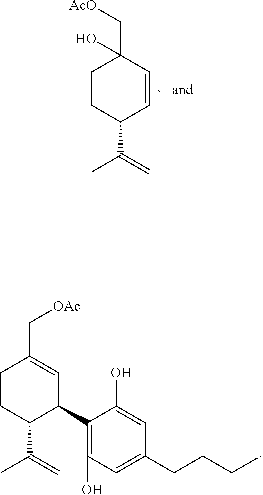

### Compound II

The invention provides a compound of formula (II):

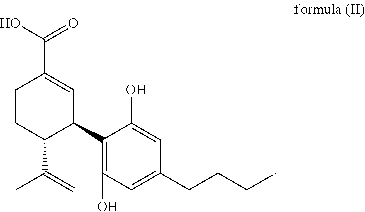

The compound of formula (II) is (1R,6R)-4′-butyl-2′,6′-dihydroxy-6-(prop-1-en-2-yl)-1,4,5,6-tetrahydro-[1,1′-biphenyl]-3-carboxylic acid. It will be referred as the compound of formula (II) or compound II herein.

### Methods of Synthesis

The invention provides a process for the production of a compound of formula (II) comprising the following steps:
i) providing 7-hydroxy-CBD-C4 (a compound of formula (I));
ii) treating 7-hydroxy-CBD-C4 with an acylation reagent and a base to give 7-hydroxy-CBD-C4 di-O-acetate;
iii) reacting 7-hydroxy-CBD-C4 di-O-acetate with an oxidising agent to give 7-formyl-CBD-C4 di-O-acetate;
iv) oxidising 7-formyl-CBD-C4 di-O-acetate, such as with sodium chlorite and monosodium phosphate, to afford 7-carboxy-CBD-C4 di-O-acetate; and
v) deprotection of carboxy-CBD-C4 di-O-acetate using a reducing agent to produce the compound of formula (II).

Suitable acetylating agents include acetic anhydride, acetyl chloride, N-succinimidyl acetate, 1-acetyl-1H-1,2,3-triazolo[4,5-b]pyridine and N-acetylaimidazole. In preferred embodiments, acetic anhydride is used.

Suitable bases include ammonium carbonate, barium carbonate, calcium carbonate, ceasium carbonate, magnesium carbonate, potassium carbonate and sodium carbonate. In preferred embodiments, caesium carbonate is used.

Suitable oxidising agents include manganese dioxide; Dess-Martin periodinane; IBX; TEMPO; TPAP; DMSO and oxalyl chloride; DMSO and carbodiimide; and SO₂·Py. In preferred embodiments, manganese dioxide is used.

Suitable reducing agents include sodium borohydride. In preferred embodiments, sodium borohydride is used.

In preferred embodiments, step i) comprises:
i) treating 3,5-dimethoxybenzyl bromide with a propyl Grignard reagent, such as n-propylmagnesium chloride, to produce 1-butyl-3,5-dimethoxybenzene;
ii) treating 1-butyl-3,5-dimethoxybenzene with a demethylation reagent, such as boron tribromide, to produce 5-butylbenzene-1,3-diol;
iii) coupling 5-butylbenzene-1,3-diol with (R)-isolimonenediol 7-O-acetate in the presence of acid, such as catalytic p-toluenesulfonic acid, to produce 7-acetoxy-CBD-C4; and
iv) treating 7-acetoxy-CBD-C4 with a reducing agent, such as sodium borohydride, to produce 7-hydroxy-CBD-C4.

### Intermediates

The invention provides an intermediate formed in the process of the production of a compound of formula (II), wherein the intermediate is selected from:

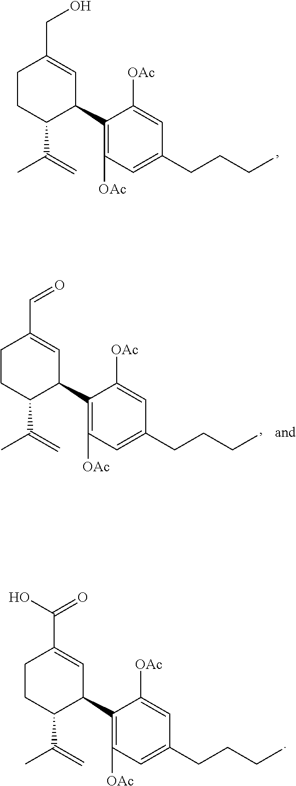

### Salts

In some embodiments, the compounds of formula (I) or (II) are provided in free base form.

Alternatively, it may be convenient or desirable to prepare, purify, and/or handle a corresponding salt of the compound, for example, a pharmaceutically-acceptable salt. Examples of pharmaceutically acceptable salts are discussed in “Pharmaceutical Salts: Properties, Selection, and Use”, 2nd Edition, 2002, Stahl and Wermuth (Eds), Wiley-VCH, Weinheim, Germany.

Accordingly, in some embodiments the compounds of formula (I) or (II) are provided as salts, for example in a protonated form together with a suitable counter anion.

Suitable counter anions include both organic and inorganic anions. Example of suitable inorganic anions include those derived from inorganic acids, including chloride (Cl⁻), bromide (Br), iodide (I⁻), sulfate (SO₄²⁻), sulfite (SO₃²⁻), nitrate (NO₃⁻), nitrite (NO₂⁻), phosphate (PO₄³⁻), and phosphite (PO₃³⁻). Examples of suitable organic anions include 2-acetoxybenzoate, acetate, ascorbate, aspartate, benzoate, camphorsulfonate, cinnamate, citrate, edetate, ethanedisulfonate, ethanesulfonate, formate, fumarate, gluconate, glutamate, glycolate, hydroxymalate, carboxylate, lactate, laurate, lactate, maleate, malate, methanesulfonate, oleate, oxalate, palmitate, phenylacetate, phenylsulfonate, propionate, pyruvate, salicylate, stearate, succinate, sulfanilate, tartarate, toluenesulfonate, and valerate. Examples of suitable polymeric organic anions include those derived from tannic acid and carboxymethyl cellulose.

Alternatively, in some embodiments the compounds of formula (I) or (II) are provided as salts, for example in a deprotonated form together with a suitable counter cation.

Suitable counter cations include both organic and inorganic cations. Examples of suitable inorganic cations include alkali metal ions such as Na⁺ and K⁺, alkaline earth cations such as Ca²⁺ and Mg²⁺, and other cations such as Al³⁺. Examples of suitable organic cations include the ammonium ion (i.e., NH₄⁺) and substituted ammonium ions (e.g., NH₃R⁺, NH₂R₂⁺, NHR₃⁺, NR₄⁺). Examples of substituted ammonium ions include those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

### Solvates

In some embodiments, the compounds of formula (I) or (II) are provided in desolvated form, for example, in dehydrated form.

Alternatively, it may be convenient or desirable to prepare, purify, and/or handle a corresponding solvate of the compound.

Accordingly, in some embodiments the compounds of formula (I) or (II) are provided in the form of a solvate (a complex of solute (e.g., compound, salt of compound) and solvent). Examples of solvates include hydrates, for example, a mono-hydrate, a di-hydrate and a tri-hydrate.

### Pharmaceutical Compositions

While it is possible for the compounds of formula (I) or (II) to be administered alone, it is preferable to administer a pharmaceutical composition (e.g., a formulation, preparation, or medicament) comprising a compound of formula (I) or (II) together with one or more other pharmaceutically acceptable ingredients.

Accordingly, the invention provides a pharmaceutical composition comprising a compound of formula (I) or (II), or a salt thereof, together with one or more pharmaceutically acceptable ingredients.

Suitable pharmaceutically acceptable ingredients (e.g. carriers, diluents, excipients, etc.) can be found in standard pharmaceutical texts, for example, Remington: The Science and Practice of Pharmacy, 20th Edition, 2000, pub. Lippincott, Williams & Wilkins; and Handbook of Pharmaceutical Excipients, 9th edition, 2020, pub. Pharmaceutical Press.

Examples of suitable pharmaceutically acceptable ingredients include pharmaceutically acceptable carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

In preferred embodiments, the pharmaceutically acceptable ingredient is selected from a carrier, an oil, a disintegrant, a lubricant, a stabilizer, a flavouring agent, an antioxidant and a diluent. Optionally, another pharmaceutically effective compound may also be included.

The pharmaceutical composition may be in any suitable form. Examples of suitable forms include liquids, solutions (e.g., aqueous, nonaqueous), suspensions (e.g., aqueous, non-aqueous), emulsions (e.g., oil-in-water, water-in-oil), syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, and aerosols.

In preferred embodiments, the pharmaceutical composition may be in the form of a tablet, a capsule, a granule, a powder for inhalation, a sprinkle, an oral solution and a suspension.

### Medical Treatment

The inventors have found that compounds of formula (I) and (II) are biologically active. The worked examples demonstrate that compounds of formula (I) and (II) display anticonvulsant activity in a mouse model of seizure. As such, the compounds of formula (I) and (II) and their salts, as well as pharmaceutical compositions comprising the compounds of formula (I) or (II), or their salts, will be useful in medical treatment.

Accordingly, the invention provides a compound of formula (I) or (II), or a salt thereof, for use in a method of treatment, for example for use in a method of treatment of the human or animal body by therapy (i.e. a method of therapy).

The invention also provides a compound of formula (I) or (II), or a salt thereof, for use as a medicament.

The invention also provides a method of treatment comprising administering to a subject in need of treatment a therapeutically effective amount of a compound of formula (I) or (II), or a salt thereof.

The invention also provides the use of a compound of formula (I) or (II), or a salt thereof, for the manufacture of a medicament.

The invention also provides use of a compound of formula (I) or (II), or a salt thereof, in a method of treatment.

### Conditions Treated

The inventors have found that the compounds of formula (I) and (II) display anticonvulsant activity in a mouse model of generalised seizure. Accordingly, the compounds of formula (I) and (II), their salts, as well as pharmaceutical compositions comprising the compounds of formula (I) or (II), or their salts, will be useful in the treatment of certain conditions associated with seizure.

Similarly, the compounds of formula (I) and (II), their salts, as well as pharmaceutical compositions comprising the compounds of formula (I) or (II), or their salts, will be useful as medicaments for treating (and in the manufacture of medicaments for treating) certain conditions associated with seizure.

In a preferred embodiment, the condition associated with seizure is epilepsy.

In one embodiment, the condition associated with seizure is generalised seizure, such as generalised seizure associated with epilepsy.

In one embodiment, the condition associated with seizure is focal-onset seizures, such as focal-onset seizure associated with epilepsy.

In one embodiment, the condition associated with seizure is tonic-clonic seizures, such as tonic-clonic seizures associated with epilepsy.

### The Subject/Patient

The method of treatment typically comprises administering a compound of formula (I) or (II), or a salt thereof, to a subject or patient.

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutan, gibbon), or a human.

The subject/patient may be any of its forms of development, for example, the subject/patient may be an infant or child.

In a preferred embodiment, the subject/patient is a mammal, more preferably a human, even more preferably an adult human.

The subject/patient may also be a non-human mammal used in laboratory research, such as a rodent. Rodents include rats, mice, guinea pigs and chinchillas.

### Routes of Administration

The method of treatment may comprise administering a compound of formula (I) or (II), or a salt thereof, to a subject by any convenient route of administration, whether systemically/peripherally or topically (i.e., at the site of desired action).

The route of administration may be oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection or infusion, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; or by implant of a depot or reservoir, for example, subcutaneously or intramuscularly.

### Dosages

The method of treatment typically comprises administering a therapeutically effective amount of a compound of formula (I) or (II), or a salt thereof, to a subject.

Appropriate dosages of the compounds of formula (I) or (II), their salts, as well as pharmaceutical compositions comprising the compounds of formula (I) or (II), or their salts, can vary from patient to patient. Determining the optimal dosage will generally involve balancing the level of therapeutic benefit against any risk or deleterious side effects. The selected dosage level will depend on a variety of factors including, but not limited to, the activity of the particular compound of formula (I) or (II), the route of administration, the time of administration, the rate of excretion of the compound, the duration of the treatment, other active agents, compounds, and/or materials used in combination, the severity of the condition, and the species, sex, age, weight, condition, general health, and prior medical history of the patient. The dosage and route of administration will ultimately be at the discretion of the clinician, although generally the dosage will be selected to achieve local concentrations at the site of action which achieve the desired effect without causing substantial harmful or deleterious side-effects.

In some embodiments, the dose of the compound of formula (I) or (II) is between 1 and 2,000 mg/day. In preferred embodiments, the dose is between 20 and 1,000 mg/day, more preferably between 50 and 500 mg/day.

Administration can be effected in one dose, continuously or intermittently (e.g., in divided doses at appropriate intervals) throughout the course of treatment. Single or multiple administrations can be carried out with the dose level and pattern being selected by the treating clinician.

### Co-Administration

The method of treatment may comprises administering a compound of formula (I) or (II), or a salt thereof, to a subject in the absence of other medications.

In some embodiments, the method of treatment comprises administering a compound of formula (I) or (II), or a salt thereof, to a subject in combination with one or more concomitant anti-epileptic drugs (AEDs).

Suitable AEDs include rufinamide; lamotrigine; topiramate; felbamate, stiripentol, clobazam and valproic acid.

Administration may be effected simultaneously or sequentially.

### Other Aspects and Embodiments

Each and every compatible combination of the embodiments described above is explicitly discloses herein, as if each and every combination was individually and explicitly recited.

Carious further aspects and embodiment of the present invention will be apparent to those skilled in the arti in view of the present disclosure.

Where used, “and/or” is to be taken as a specific disclosure of each of the relevant components or features alone as well as a specific disclosure of the combination of the components or features. For example, “A and/or B” is to be taken as specific disclosure of each of i) A, ii) B, and ii) A and B, just as if each were set out individually.

Unless context dictates otherwise, the descriptions and definitions of the features set out above are not limited to any particular aspect or embodiment of the invention and apply equally to all aspects ad embodiments which are described.

### Definitions

Definitions of some of the terms used to describe the invention are detailed below.

The compounds described in the present application are listed below.

| Compound | Abbreviation | Short Name | Structure |
|---|---|---|---|
| Compound I | 7-OH-CBD-C4 | 7-hydroxy-cannabidiol-C4 | 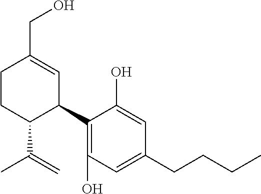 [SMILES: C=C(C)[C@@H]1CCC(CO)=C[C@H]1c1c(O)cc(CCCC)cc1O] |
| Compound II | 7-COOH-CBD-C4 | 7-carboxy-cannabidiol-C4 | 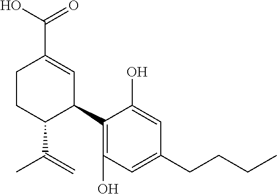 [SMILES: C=C(C)[C@@H]1CCC(C(=O)O)=C[C@H]1c1c(O)cc(CCCC)cc1O] |

Epilepsy is considered to be a disease of the brain defined by any of the following conditions: (1) At least two unprovoked (or reflex) seizures occurring >24 h apart; (2) one unprovoked (or reflex) seizure and a probability of further seizures similar to the general recurrence risk (at least 60%) after two unprovoked seizures, occurring over the next 10 years; (3) diagnosis of an epilepsy syndrome (A practical clinical definition of epilepsy by the International League Against Epilepsy (ILAE), 2014).

The term “focal seizure” (“focal onset seizure”) refers to seizures originating within networks limited to one hemisphere. They may be discretely localized or more widely distributed. Focal seizures may originate in subcortical structures (Operational Classification of Seizure Types by the ILAE, 2017).

The term “generalized seizure” (“generalized onset seizures”) refers to seizures conceptualized as originating at some point within the brain and rapidly engaging bilaterally distributed networks (Operational Classification of Seizure Types by the ILAE, 2017).

The term “pharmaceutically acceptable” pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each ingredient (e.g. carrier, diluent, excipient, etc.) must also be “acceptable” in the sense of being compatible with the other ingredients of the composition.

The term “therapeutically-effective amount” pertains to that amount of a compound, or a material, composition or dosage form comprising a compound, which is effective for producing some desired therapeutic effect, commensurate with a reasonable benefit/risk ratio, when administered in accordance with a desired treatment regimen.

A “tonic-clonic seizure” occurs in two phases, a tonic phase typically involving muscle stiffening and loss of consciousness, and a clonic phase typically involving rhythmically jerking of the limbs.

The human dose equivalent (HED) can be estimated using the following formula:

$$H E D ( mg / kg ) = Animal dose ( mg / kg ) \times \frac{Animal {K}_{m}}{Human {K}_{m}}$$

The Kₘ for a rat is 6 and the Kₘ for a human is 37. Thus, for a human of approximately 60 Kg a 200 mg/Kg dose in rat would equate to a human daily dose of about 2,000 mg.

### WORKED EXAMPLES

Certain aspects and embodiments of the invention will not be illustrated by way of example and with reference to the figures described above.

### Example 1: Synthetic Production Method for Compounds I and II

This example describes a novel method of synthesis which was used to produce novel compounds which demonstrated pharmacological activity. Scheme 1 below describes the four stages of the reaction which was used to produce the novel compounds, formed via a number of intermediates.

Scheme 1 below shows the synthetic route of Compound I in two steps and synthetic route of Compound II in three steps. Step 1 produces 5-butylbenzene-1,3-diol (compound c), required for the preparation of Compound I. Step 2 uses compound c of Step 1 to produce Compound I. Step 3 uses Compound I to produce Compound II.

To summarise, in step 1, treatment of 3,5-dimethoxybenzyl bromide with n-propylmagnesium chloride in the presence of catalytic di-lithium tetrachlorocuprate provides 1-butyl-3,5-dimethoxybenzene. Removal of the phenolic methyl ethers with boron tribromide in dichloromethane gives 5-butylbenzene-1,3-diol after chromatographic purification.

In step 2, the synthesis of Compound I is achieved by coupling (R)-isolimonenediol 7-O-acetate with 5-butylbenzene-1,3-diol in the presence of catalytic p-toluenesulfonic acid in dichloromethane to afford 7-acetoxy-CBD-C4. Removal of the acetate group using sodium borohydride gives Compound I after chromatographic purification.

In step 3, treatment of Compound I with acetic anhydride and caesium carbonate selectively protects the phenolic groups to give the diacetate. Oxidation of the allylic alcohol with manganese dioxide gives the corresponding aldehyde. This aldehyde is further oxidised under Pinnick conditions to afford 7-carboxy-CBD-C4 di-O-acetate after purification. Deprotection using sodium borohydride gives Compound II.

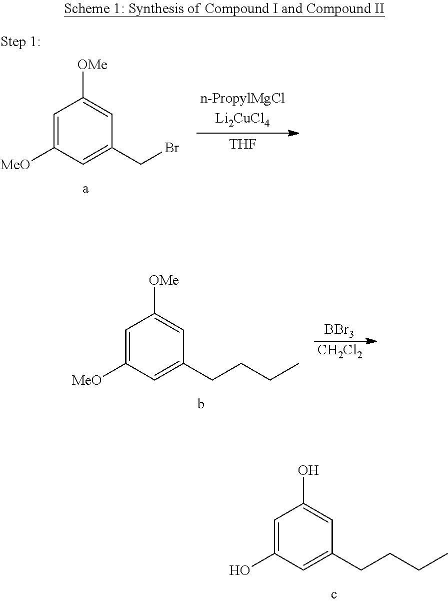

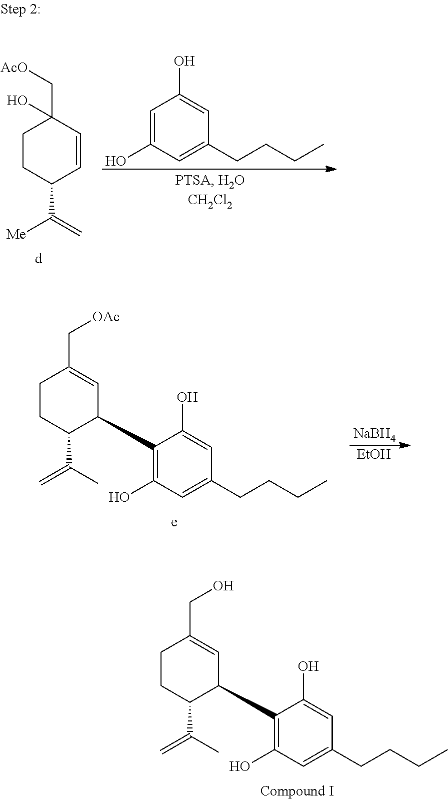

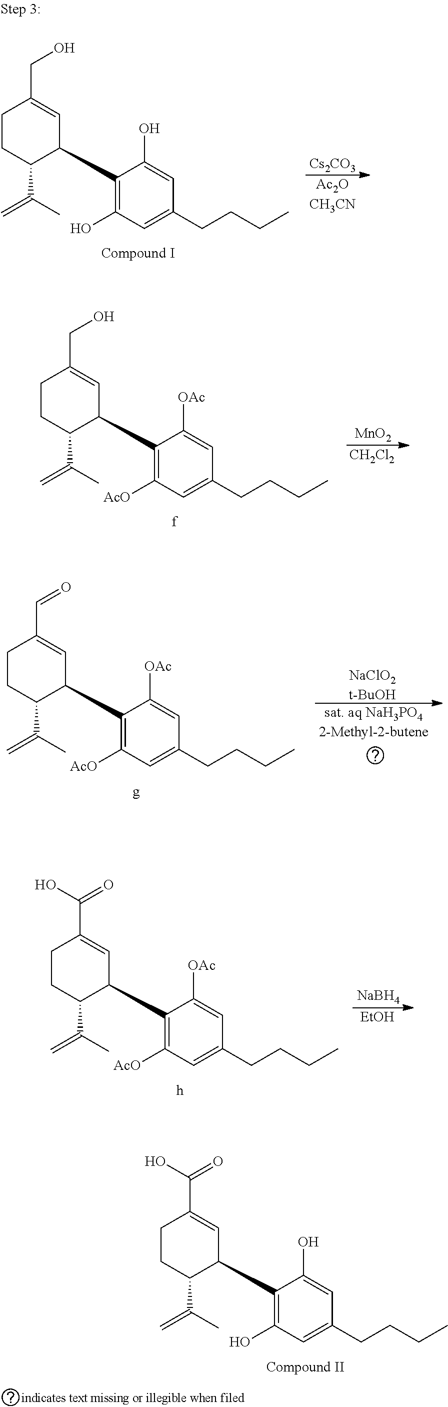

| Compound | Name |
|---|---|
| a | 3,5-dimethoxybenzyl bromide |
| b | 1-butyl-3,5-dimethoxybenzene |
| c | 5-butylbenzene-1,3-diol |
| d | (R)-isolimonenediol 7-O-acetate |
| e | 7-acetoxy-CBD-C4 |
| f | 7-hydroxy-CBD-C4 di-O-acetate |
| g | 7-formyl-CBD-C4 di-O-acetate |
| h | 7-carboxy-CBD-C4 di-O-acetate |

### Step 1: Production of Compound c

To a solution of 3,5-dimethoxybenzylbromide (1.00 g, 4.33 mmol) and 0.1 M di-5 lithium copper tetrachloride in tetrahydrofuran (4.3 mL, 0.43 mmol) at −78° C. was added dropwise a solution of 2 M propylmagnesium chloride in diethyl ether (5.4 mL, 10.8 mmol) under nitrogen. The reaction mixture was allowed to warm to room temperature and was stirred for 2.5 h. The resulting mixture was quenched with saturated aqueous ammonium chloride (30 mL) and diluted with diethyl ether (30 mL). The two layers were separated, and the aqueous phase was extracted with diethyl ether (2×30 mL). The combined organic layers were washed with water (60 mL), saturated brine (60 mL), dried (MgSO₄) and concentrated to give a pale brown oil. This crude material was purified using a Biotage Isolera automated chromatography system under normal phase conditions (silica column, gradient 0→12% diethyl ether in petrol) with detection at 254 nm to give 1-butyl-3,5-dimethoxybenzene (0.68 g, 81%), as a colourless oil.

To 1-butyl-3,5-dimethoxybenzene (0.68 g, 3.48 mmol) in anhydrous dichloromethane (11 mL) at −78° C. was added 1 M boron tribromide in dichloromethane (10.4 mL, 10.4 mmol) and the mixture was stirred at −78° C. for 10 min followed by 4.5 h at room temperature. The resulting mixture was cooled to 0° C. and cautiously quenched with methanol (5 mL) and water (15 mL) The layers were separated, and the aqueous phase was extracted with dichloromethane (2×10 mL). The combined organic layers were washed with saturated brine (30 mL), dried (MgSO₄) and concentrated to give an orange oil. This residual oil was purified using a Biotage Isolera automated chromatography system under normal phase conditions (silica column, gradient of 0→60% ethyl acetate in petrol) with detection at 254 nm to give 5-butylbenzene-1,3-diol (0.55 g, 96%), as a pale yellow oil.

### Step 2: Production of Compound I

To 5-butylbenzene-1,3-diol (0.50 g, 3.00 mmol) in anhydrous dichloromethane (100 mL) at 0° C. was added p-toluenesulfonic acid monohydrate (286 mg, 1.50 mmol) followed by ((1S,4R)-1-hydroxy-4-(prop-1-en-2-yl)cyclohex-2-en-1-yl)methyl acetate (0.63 g, 3.00 mmol) in anhydrous dichloromethane (7 mL) dropwise and the resulting solution was stirred at room temperature for 40 min. The reaction was quenched with saturated aqueous sodium hydrogen carbonate (100 mL) and the layers were separated. The aqueous layer was extracted with dichloromethane (3×40 mL) and the combined organic layers were washed with saturated brine (100 mL), dried (MgSO₄) and concentrated. The residual material was purified using a Biotage Isolera automated chromatography system under normal phase conditions (silica column, gradient of 5->35% diethyl ether in petrol) with detection at 254 nm to afford ((1R,6R)-4′-butyl-2′,6′-dihydroxy-6-(prop-1-en-2-yl)-1,4,5,6-tetrahydro-[1,1′-biphenyl]-3-yl)methyl acetate (295 mg, 27%), as a pale yellow oil.

To a solution ((1R,6R)-4′-butyl-2′,6′-dihydroxy-6-(prop-1-en-2-yl)-1,4,5,6-tetrahydro-[1,1′-biphenyl]-3-yl)methyl acetate (295 mg, 0.82 mmol) in anhydrous ethanol (41 mL) was added sodium borohydride (47 mg, 1.23 mmol) and the mixture was heated at 80° C. for 1 h. The reaction mixture was allowed to cool to room temperature, water (30 mL) was added and the solution was concentrated to remove the ethanol. The resulting mixture was extracted with ethyl acetate (4×20 mL) and the combined organic layers were washed with saturated brine (50 mL), dried (MgSO₄) and concentrated to give a pale orange oil. This residual material was purified using a Biotage Isolera automated chromatography system under normal phase conditions (silica column, gradient of 10→55% ethyl acetate in petrol) with detection at 254 nm to afford Compound I, (1′R,2′R)-4-butyl-5′-(hydroxymethyl)-2′-(prop-1-en-2-yl)-1′,2′,3′,4′-tetrahydro-[1,1′-biphenyl]-2,6-diol (157 mg, 61%), as an off-white solid.

### Step 3: Production of Compound II

Compound I, (1′R,2′R)-4-Butyl-5′-(hydroxymethyl)-2′-(prop-1-en-2-yl)-1′,2′,3′,4′-tetrahydro-[1,1′-biphenyl]-2,6-diol (508 mg, 1.61 mmol) was dissolved in anhydrous acetonitrile (27 mL), caesium carbonate (1.10 g, 3.37 mmol) was added and the resulting dark purple heterogeneous mixture was stirred for 5 min. Acetic anhydride (329 mg, 300 μL, 3.22 mmol) was added to this mixture and stirring was continued for 1.5 h at room temperature. Water (100 mL) was added and the resulting mixture was extracted with ethyl acetate (3×100 mL). The combined organic layers were washed with saturated brine (200 mL), dried (MgSO₄) and concentrated to give a brown oil. This residual material was purified using a Biotage Isolera automated chromatography system under normal phase conditions (silica column, gradient of 9→65% ethyl acetate in petrol) with detection at 254 nm to afford (1′R,2′R)-4-butyl-5′-(hydroxymethyl)-2′-(prop-1-en-2-yl)-1′,2′,3′,4′-tetrahydro-[1,1′-biphenyl]-2,6-diyl diacetate (387 mg, 60%), as an orange oil.

To a solution of (1′R,2′R)-4-butyl-5′-(hydroxymethyl)-2′-(prop-1-en-2-yl)-1′,2′,3′,4′-tetrahydro-[1,1′-biphenyl]-2,6-diyl diacetate (387 mg, 0.97 mmol) in anhydrous dichloromethane (20 mL) was added manganese (IV) oxide (1.68 g, 19.3 mmol) and the resulting black suspension was heated at reflux for 2 h. The suspension was filtered through Celite and concentrated to give (1′R,2′R)-4-butyl-5′-formyl-2′-(prop-1-en-2-yl)-1′,2′,3′,4′-tetrahydro-[1,1′-biphenyl]-2,6-diyl diacetate (229 mg, 77%), as a yellow oil.

To a solution of (1′R,2′R)-4-butyl-5′-formyl-2′-(prop-1-en-2-yl)-1′,2′,3′,4′-tetrahydro-[1,1′-biphenyl]-2,6-diyl diacetate (229 mg, 0.57 mmol) in tert-butanol (14 mL) and 2-methyl-2-butene (1.02 g, 1.54 mL, 14.5 mmol) was added saturated aqueous sodium dihydrogen phosphate (0.78 mL). Sodium chlorite (80%, 229 mg, 2.53 mmol) was added in small portions to the reaction mixture and stirring was continued overnight at room temperature. Water (50 mL) was added and the mixture was extracted with ethyl acetate (4×30 mL). The combined organic layers were washed with saturated brine (60 mL), dried (MgSO₄) and concentrated to give (1R,6R)-2′,6′-diacetoxy-4′-butyl-6-(prop-1-en-2-yl)-1,4,5,6-tetrahydro-[1,1′-biphenyl]-3-carboxylic acid (294 mg, quant.), as a yellow oil.

To a solution of (1R,6R)-2′,6′-diacetoxy-4′-butyl-6-(prop-1-en-2-yl)-1,4,5,6-tetrahydro-[1,1′-biphenyl]-3-carboxylic acid (294 mg, 0.71 mmol) in anhydrous ethanol (18 mL) was added sodium borohydride (40 mg, 1.06 mmol) and the reaction mixture was heated at 80° C. for 2 h. The resulting mixture was allowed to cool to room temperature, water (30 mL) was added and the solution was concentrated to remove the ethanol. The mixture was extracted with ethyl acetate (4×20 mL) and the combined organic layers were washed with saturated water (20 mL), dried (MgSO₄) and concentrated to give Compound II, (1R,6R)-4′-butyl-2′,6′-dihydroxy-6-(prop-1-en-2-yl)-1,4,5,6-tetrahydro-[1,1′-biphenyl]-3-carboxylic acid (195 mg, 83%), as an off-white solid.

### Example 2: Evaluation of Novel Compounds for Anticonvulsant Activity Using the Maximal Electroshock Seizure Threshold (MEST) Test in the Mouse Using Minimal Sample Size (Mini-MEST)

The efficacy of the novel compounds according to Formula I and Formula II were tested in a novel mouse model of generalised seizure, the mini-MEST (maximal electroshock seizure threshold) test, which uses lower n numbers than typically used.

The maximal electroshock seizure threshold (MEST) test is widely utilized preclinically to evaluate pro- or anti-convulsant properties of test compounds (Loscher et al., 1991).

In the MEST test the ability of a drug to alter the seizure threshold current required to induce hind limb tonic extensor convulsions is measured according to an “up and down” method of shock titration (Kimball et al., 1957). An increase in seizure threshold is indicative of anti-convulsant effect. Antiepileptic drugs including the sodium channel blockers (e.g. lamotrigine) with clinically proven efficacy against generalised tonic-clonic seizures all exhibit anti-convulsant properties in this test in the mouse.

Conversely, a reduction in seizure threshold is indicative of a pro-convulsant effect as observed with known convulsant agents such as picrotoxin.

The ability of a test compound to alter the stimulus intensity, expressed as current (mA), required to induce the presence of tonic hind limb extensor convulsions, is assessed in the MEST. The outcome of the presence (+) or absence (0) of tonic hind limb extensor convulsions observed from a current to produce tonic hind limb extension in 50% of animals in the treatment group (CC₅₀) determines the seizure threshold for the treatment group and the effects were then compared to the CC₅₀ of the vehicle control group.

### Methods

### Study Details:

Naïve mice were acclimatised to the procedure room in their home cages for up to 7 days, with food and water available ad libitum.

All animals were weighed at the beginning of the study and randomly assigned to treatment groups based on a mean distribution of body weight across groups. All animals were dosed at 10 mL/kg via intraperitoneal (i.p) injection, with either vehicle, test compound at 100 mg/kg (Compound I) or 150 mg/kg (Compound II), or diazepam at 2.5 mg/kg.

Animals were individually assessed for the production of a tonic hind limb extensor convulsion at 30 min post-dose for vehicle, test compound and diazepam, from a single electroshock.

The first animal within a treatment group was given a shock at the expected or estimated CC₅₀ current. For subsequent animals, the current was lowered or raised depending on the convulsions outcome from the preceding animal in log scale intervals.

Data generated from each treatment group were used to calculate the CC₅₀±SEM values for the treatment group.

### Test Compounds:

Vehicle: (5% ethanol, 10% solutol in 85% Saline) was prepared as follows: 1 mL of ethanol, 2 mL of solutol were warmed to 60° ° C., in 17 mL of saline (1:2:17).

Positive control: diazepam was used at 2.5 mg/kg.

The test compounds, described herein as Compound I and Compound II, are as shown as Formula I and Formula II respectively. Test compounds were administered at 100 mg/kg (i.p.) for Compound I and 150 mg/kg (i.p.) for Compound II in a 1:2:17 ethanol:solutol:0.9% saline formulation.

### Sample Collection:

Each animal was humanely killed immediately after production of a convulsion by destruction of the brain from striking the cranium, followed by the confirmation of permanent cessation of the circulation from decapitation under The Humane Killing of Animals under Schedule 1 to the Animals (Scientific Procedures) Act 1986. Terminal blood and brain collection were performed following decapitation.

Blood was collected in Lithium-heparin tubes and centrifuged at 4ºC for 10 minutes at 1500× g. The resulting plasma was removed (>100 μL) and split into 2 aliquots of 0.5 mL Eppendorf tubes containing 10 μL of ascorbic acid (100 mg/mL) for stabilisation. Brains were removed, washed in saline and halved. Each half was placed into separate 2 mL screw cap cryovials, weighed and frozen on cardice.

### Statistical Analysis

The data for each treatment group were recorded as the number of +'s and 0's at each current level employed and this information is then used to calculate the CC₅₀ value (current required for 50% of the animals to show seizure behaviour)±standard error.

Test compound effects were also calculated as percentage change in CC₅₀ from the vehicle control group.

Significant difference between drug-treated animals and controls were assessed according to Litchfield and Wilcoxon (1949).

### Results

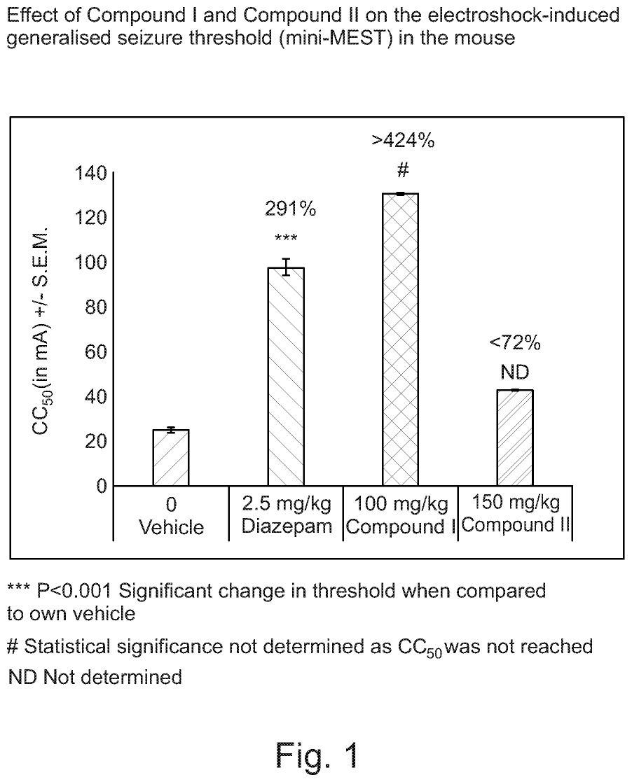

FIG. 1 and Table 1 describe the data produced in this experiment.

In the vehicle group, the CC₅₀ value was calculated to be 25.0 mA.

In the diazepam (2.5 mg/kg) treated group, administered i.p. 30 minutes before the test, the CC₅₀ value was 97.8 mA. This result was statistically significant (p<0.001) compared to vehicle control.

Compound I, administered i.p. 30 minutes before the test, produced a clear increase in seizure threshold as compared to vehicle, with CC₅₀>131 mA for 100 mg/kg; an exact value was not calculated as a “+” tonic hindlimb convulsion was not seen within the 6 animals tested. Although CC₅₀ was not determined, Compound I showed a clear increase in seizure threshold in the mini-MEST. Clear activity of Compound I was demonstrated as animals in this treatment group did not have any convulsions.

Compound II, also administered i.p. 30 minutes before the test, did not produce as high an increase in seizure threshold as Compound I, with CC₅₀<43 mA for 150 mg/kg.

Such data are indicative that these compounds will be of therapeutic benefit.

TABLE 1
Evaluation of effect of Compound I and
Compound II in the mini-MEST test

| | Dose | Test time | | | | % change |
|---|---|---|---|---|---|---|
| | (mg/ | post dose | | CC₅₀ +/− | Signif- | from |
| Treatment | kg) | (min) | N | SEM | icance | vehicle |
| Vehicle | — | 30 | 6 | 25.0 ± 1.1 | — | — |
| Diazepam | 2.5 | 30 | 6 | 97.8 ± 3.7 | P < 0.001 | 291% |
| Compound I | 100 | 30 | 6 | >131 | # | >424% |
| Compound II | 150 | 30 | 6 | <43 | ND | <72% |

# Statistical significance not determined as CC₅₀ was not reached
ND Not determined

### Conclusions

This data produced using the mini-MEST model demonstrates a therapeutic effect for these compounds. In particular Compound I demonstrated a strong therapeutic effect, with CC₅₀ increasing by more than 424% compared to vehicle control, a percentage change that is even higher than positive control.

This data is significant as it provides heretofore unknown evidence that these novel compounds may be of therapeutic value.

### Example 3: Bioanalysis of Novel Compounds

Bioanalysis experiments were carried out to determine the quantitative measurement of Compound I and Compound II in plasma and brain.

### Methods

Six mice were dosed with Compound I at 100 mg/kg and another six with Compound II at 150 mg/kg via intraperitoneal (i.p) injection. Sample analysis was performed 30 min post-dose using a sample volume of 40 μL. Following addition of internal standard (in acetonitrile) and Isopropanol samples were protein precipitated by addition of acetonitrile. Samples were centrifuged and supernatant transferred to a clean 96-well plate (with addition of Tween to minimise non-specific binding to plate materials), evaporated to dryness then reconstituted in suitable solvent. The extracts quantified by ultraperformance liquid chromatography-tandem mass spectrometry (UPLC-MS/MS).

### Results

Table 2 shows the data produced in this experiment. High concentrations of Compound I and Compound II were detected in both plasma and brain.

TABLE 2
Concentrations of Compound I and
Compound II in plasma and brain

| | | | Plasma | | |
|---|---|---|---|---|---|
| | Dose | | Conc. | Mean Plasma | |
| Treatment | (mg/kg) | Location | (nM) | Conc. (nM) | SD |
| Compound | 100 | Plasma | NS | 250898 | 7929 |
| I | | | 242732 | | |
| | | | 263691 | | |
| | | | 252278 | | |
| | | | 247131 | | |
| | | | 248660 | | |
| Compound | 100 | Brain | 130750 | 121602 | 9270 |
| I | | | 122248 | | |
| | | | 113886 | | |
| | | | 107349 | | |
| | | | 125595 | | |
| | | | 129784 | | |
| Compound | 150 | Plasma | 959506 | 1053514 | 134194 |
| II | | | 856643 | | |
| | | | 1008984 | | |
| | | | 1144841 | | |
| | | | 1209706 | | |
| | | | 1141405 | | |
| Compound | 150 | Brain | 15676 | 19849 | 3618 |
| II | | | 22356 | | |
| | | | 22956 | | |
| | | | 15408 | | |
| | | | 23332 | | |
| | | | 19363 | | |

NS: No sample

### Conclusions

This data confirms presence of Compound I and Compound II in plasma and brain at quantifiable levels

1. A compound of formula (I), or a salt thereof:

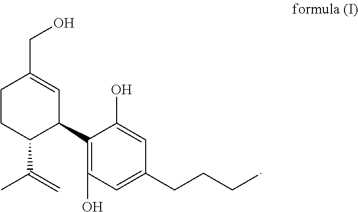

2. The compound of claim 1 as a pure, isolated or synthetic compound.

3. A pharmaceutical composition comprising a compound of formula (I) or a salt thereof.

4. The pharmaceutical composition of claim 3 in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.

5. The pharmaceutical composition of claim 3 or claim 4 comprising one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

6. A compound of formula (I), or a salt thereof, for use a medicament.

7. The compound for use of claim 6, wherein the medicament is a medicament for treating epilepsy.

8. The compound for use of claim 6 or claim 7, wherein the medicament is a medicament for treating generalised seizures, focal-onset seizures, or tonic-clonic seizures.

9. A compound of formula (I), or a salt thereof, for use in a method of treatment.

10. The compound for use of claim 9, wherein the method of treatment is a method of treating epilepsy.

11. The compound for use of claim 9 or claim 10, wherein the method of treatment is a method of treating generalised seizures, focal-onset seizures, or tonic-clonic seizures.

12. The compound for use of any of claims 9 to 11, wherein the compound is used in combination with one or more concomitant anti-epileptic drugs (AEDs).

13. The compound for use of claim 12, wherein the AEDs are selected from rufinamide; lamotrigine; topiramate; felbamate, stiripentol, clobazam and valproic acid.

14. The compound for use of any of claims 9 to 13, wherein the dose of the compound is between 1 and 2,000 mg/day, such as between 20 and 1,000 mg/day, such as between 50 and 500 mg/day.

15. A method of treatment comprising administering to a subject in need of treatment a therapeutically effective amount of a compound of formula (I) or a salt thereof.

16. The method of treatment of claim 15, wherein the method of treatment is a method of treating epilepsy.

17. The method of treatment of claim 15 or 16, wherein the method of treatment is a method of treating generalised seizures, focal-onset seizures, or tonic-clonic seizures.

18. A process for the production of a compound of formula (I) comprising the following steps:
i) treating 3,5-dimethoxybenzyl bromide with a propyl Grignard reagent to produce 1-butyl-3,5-dimethoxybenzene;
ii) treating 1-butyl-3,5-dimethoxybenzene with a demethylation reagent to produce 5-butylbenzene-1,3-diol;
iii) coupling 5-butylbenzene-1,3-diol with (R)-isolimonenediol 7-O-acetate in the presence of acid to produce 7-acetoxy-CBD-C4; and
iv) treating 7-acetoxy-CBD-C4 with a reducing agent to produce the compound of formula (I).

19. The method of claim 18, wherein the Grignard reagents is selected from n-propylmagnesium chloride and n-propylmagnesium bromide, such as n-propylmagnesium chloride.

20. The method of claim 18 or claim 19, wherein the demethylation reagent is selected from boron tribromide, beryllium dichloride, aluminium trichloride, trimethylsilyliodide and pyridine hydrochloride; such as boron tribromide.

21. The method of any of claims 18 to 20, wherein the acid is selected from p-toluenesulfonic acid and aluminium trichloride, such as catalytic p-toluenesulfonic acid.

22. The method of any of claims 18 to 21, wherein the reducing agent is selected from lithium aluminium hydride, sodium bis(2-methoxyethoxy)aluminium hydride (red-Al), diborane and sodium borohydride; such as sodium borohydride.

23. An intermediate formed in the process of the production of a compound of formula (I), wherein the intermediate is selected from:

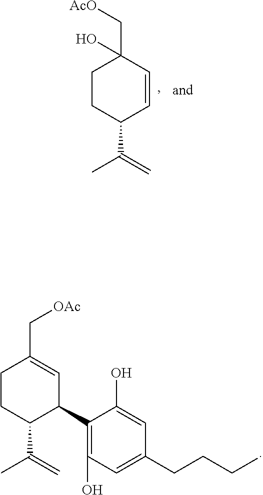

24. A compound of formula (II) or a salt thereof:

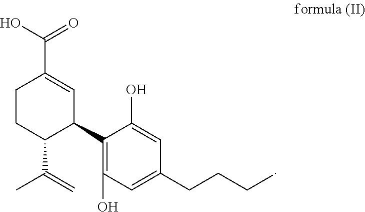

25. The compound of claim 24 as a pure, isolated or synthetic compound.

26. A pharmaceutical composition comprising a compound of formula (II) or a salt thereof.

27. The pharmaceutical composition of claim 26 in a form selected from a liquid, a solution, a suspension, an emulsion, a syrup, an electuary, a mouthwash, a drop, a tablet, a granule, a powder, a lozenge, a pastille, a capsule, a cachet, a pill, an ampoule, a bolus, a suppository, a pessary, a tincture, a gel, a paste, an ointment, a cream, a lotion, an oil, a foam, a spray, and an aerosol.

28. The pharmaceutical composition of claim 26 or claim 27 comprising one or more ingredients selected from carriers, diluents, excipients, adjuvants, fillers, buffers, binders, disintegrants, preservatives, antioxidants, lubricants, stabilisers, solubilisers, surfactants (e.g., wetting agents), masking agents, colouring agents, flavouring agents, and sweetening agents.

29. A compound of formula (II), or a salt thereof, for use a medicament.

30. The compound for use of claim 29, wherein the medicament is a medicament for treating epilepsy.

31. The compound for use of claim 29 or claim 30, wherein the medicament is a medicament for treating generalised seizures, focal-onset seizures, or tonic-clonic seizures.

32. A compound of formula (II), or a salt thereof, for use in a method of treatment.

33. The compound for use of claim 32, wherein the method of treatment is a method of treating epilepsy.

34. The compound for use of claim 32 or claim 33, wherein the method of treatment is a method of treating generalised seizures, focal-onset seizures, or tonic-clonic seizures.

35. The compound for use of any of claims 32 to 34 wherein the compound is used in combination with one or more concomitant anti-epileptic drugs (AEDs).

36. The compound for use of claim 35, wherein the AEDs are selected from rufinamide; lamotrigine; topiramate; felbamate, stiripentol, clobazam and valproic acid.

37. The compound for use of any of claims 32 to 36, wherein the dose of the compound is between 1 and 2,000 mg/day, such as between 20 and 1,000 mg/day, such as between 50 and 500 mg/day.

38. A method of treatment comprising administering to a subject in need of treatment a therapeutically effective amount of a compound of formula (I) or a salt thereof.

39. The method of treatment of claim 38, wherein the method of treatment is a method of treating epilepsy.

40. The method of treatment of claim 38 or 39, wherein the method of treatment is a method of treating generalised seizures, focal-onset seizures, or tonic-clonic seizures.

41. A process for the production of a compound of formula (II) comprising the following steps:
i) providing 7-hydroxy-CBD-C4 (a compound of formula (I));
treating 7-hydroxy-CBD-C4 with an acylation reagent and a base to give 7-hydroxy-CBD-C4 di-O-acetate;
iii) reacting 7-hydroxy-CBD-C4 di-O-acetate with an oxidising agent to give 7-formyl-CBD-C4 di-O-acetate;
iv) oxidising 7-formyl-CBD-C4 di-O-acetate, such as with sodium chlorite and monosodium phosphate, to afford 7-carboxy-CBD-C4 di-O-acetate; and
v) deprotection of carboxy-CBD-C4 di-O-acetate using a reducing agent to produce the compound of formula (II).

42. The method of claim 41, wherein the acylation reagent is selected from acetic anhydride, acetyl chloride, N-succinimidyl acetate, 1-acetyl-1H-1,2,3-triazolo[4,5-b]pyridine and N-acetylaimidazole; such as acetic anhydride.

43. The method of claim 41 or 42, wherein the base is selected from ammonium carbonate, barium carbonate, calcium carbonate, ceasium carbonate, magnesium carbonate, potassium carbonate and sodium carbonate; such as caesium carbonate.

44. The method of any of claims 41 to 43, wherein the oxidising agent is selected from manganese dioxide; Dess-Martin periodinane; IBX; TEMPO; TPAP; DMSO and oxalyl chloride; DMSO and carbodiimide; and SO₂·Py; such as manganese dioxide.

45. The method of any of claims 41 to 44, wherein the reducing agent is selected from sodium borohydride.

46. The method of any of claims 41 to 45, wherein step i) comprises:
i) treating 3,5-dimethoxybenzyl bromide with a propyl Grignard reagent, such as n-propylmagnesium chloride, to produce 1-butyl-3,5-dimethoxybenzene;
ii) treating 1-butyl-3,5-dimethoxybenzene with a demethylation reagent, such as boron tribromide, to produce 5-butylbenzene-1,3-diol;
iii) coupling 5-butylbenzene-1,3-diol with (R)-isolimonenediol 7-O-acetate in the presence of acid, such as catalytic p-toluenesulfonic acid, to produce 7-acetoxy-CBD-C4; and
iv) treating 7-acetoxy-CBD-C4 with a reducing agent, such as sodium borohydride, to produce 7-hydroxy-CBD-C4.

47. An intermediate formed in the process of the production of a compound of formula (II), wherein the intermediate is selected from: [chemical structure]
